(19) United States
(12) Patent Application Publication
(10) Pub. No.: US 2025/0242006 A1
(43) Pub. Date: Jul. 31, 2025

(54) DRY POWDER COMPOSITIONS COMPRISING EUKARYOTIC CELLS AND METHOD OF THEIR MANUFACTURE AND USE

(71) Applicant: BOARD OF REGENTS, THE UNIVERSITY OF TEXAS SYSTEM, Austin, TX (US)

(72) Inventors: Zhengrong CUI, Austin, TX (US); Mahmoud S. HANAFY, Jr., Austin, TX (US); Robert O. WILLIAMS, III, Austin, TX (US)

(21) Appl. No.: 18/705,061

(22) PCT Filed: Oct. 27, 2022
(86) PCT No.: PCT/US2022/078792

§ 371 Date: Apr. 26, 2024

Related U.S. Application Data
(60) Provisional application No. 63/272,607, filed on Oct. 27, 2021.

(51) Int. Cl. A61K 39/00; A61K 9/16; A61K 9/19; A61K 31/352

(52) U.S. Cl. CPC A61K 39/0011; A61K 9/1623; A61K 9/1635; A61K 9/19; A61K 31/352; A61K 2039/5152

(57) ABSTRACT

Dry powder compositions comprising eukaryotic cells and methods for the manufacture of such dry powders are provided. In some aspects, these dry powder compositions may further comprise a sugar and sugar alcohol and an antioxidant or a polymer. These compositions may show increased viability compared to the dry powder compositions without a sugar or sugar alcohol and an antioxidant or polymer.

This application claims the benefit of priority to U.S. Provisional Application No. 63/272,607, filed on Oct. 27, 2021, the entire contents of which are hereby incorporated by reference.

### BACKGROUND

### 1. Field

The present disclosure relates generally to the field of pharmaceutical formulation, biologics and the manufacture of the same. More particularly, it concerns dry powder compositions that include eukaryotic cells and methods of preparing powder compositions, such as by thin-film freezing drying.

### 2. Description of Related Art

Eukaryotic cells are commonly used in biomedical research. Recently, cell-based therapy (e.g., ABECMA) has proven effective in treating cancers. Successful commercial and clinical applications of cells and cell-based therapeutics require complicated storage and transport/distribution considerations. Unfortunately, significant cell damage occurs during storage and/or distribution if the cells are not sufficiently cryopreserved. Therefore, cells must be stored at extremely low temperatures, such as below −135° C., because the cells are frozen in the presence of membrane permeable cryoprotectants such as dimethyl sulfoxide (DMSO), which enables the freezing of cells without intracellular ice crystal formation and the storage of cells in a glassy amorphous state. However, those cryoprotectants have extremely low glass transition temperature, generally below −83° C., necessitating nitrogen vapor phase cryopreservation of cell products. Cells that are frozen in the presence of DMSO may be shipped frozen with dry ice and then thawed for propagation. cGMP-manufactured cell-based therapeutics, however, are supplied as a DMSO-containing frozen suspension and shipped to the cell lab or clinical pharmacy associated with special infusion centers in the vapor phase of a liquid nitrogen shipper. Upon thawing, a cell therapy product must be infused to the intended patient within 30-60 min, although it may be stored at room temperature for 2-3 hours. Severe side effects and even fatalities have been linked to the use of DMSO. Furthermore, shipping with either dry ice or in a liquid nitrogen shipper is hazardous and costly. Likewise, new formulations and methodologies are required to provide efficient ways to delivery cells to laboratories and patients in need.

### SUMMARY

In some embodiments, the present disclosure provides dry powder compositions comprising one or more eukaryotic cells with a sugar or sugar alcohol and an antioxidant or a polymer. In some aspects, the dry powder compositions comprise one or more eukaryotic cells with a sugar or sugar alcohol, an antioxidant, or a polymer.

In some aspects, the present disclosure provides pharmaceutical compositions comprising:
(A) one or more eukaryotic cells;
(B) a sugar or sugar alcohol; and
(C) an antioxidant or a polymer;
wherein the pharmaceutical composition is formulated as a dry powder.

In some embodiments, the eukaryotic cell is an animal cell such as a mammalian cell. In some embodiments, the eukaryotic cell is a mouse cell or a human cell. In some embodiments, the eukaryotic cell is a human cell. In some embodiments, the human cell is an abnormal cell such as a cancerous cell. In other embodiments, the human cell is a normal cell.

In some embodiments, the sugar or sugar alcohol is a sugar. In some embodiments, the sugar is a disaccharide. In some embodiments, the sugar is a non-reducing disaccharide. In some embodiments, the sugar is trehalose, sucrose, or maltose. In some embodiments, the sugar is trehalose.

In some embodiments, the antioxidant is a flavonoid such as a polyphenol. In some embodiments, flavonoid is a catechin. In some embodiments, the flavonoid is a gallate ester. In some embodiments, the flavonoid is epigallocatechin gallate. In other embodiments, the antioxidant is a vitamin such as vitamin C or vitamin C derivative. In some embodiments, the vitamin is L-ascorbic acid. In some embodiments, the vitamin is vitamin E or a vitamin E derivative such as a D-α-tocopherol succinate or a PEGylated version thereof. In some embodiments, the vitamin E derivative is Trolox. In some embodiments, the vitamin E derivative is PEGylated D-α-tocopherol succinate. In some embodiments, the vitamin is D-α-tocopherol PEG1000 succinate. In other embodiments, the antioxidant is a tripeptide such as glutathione.

In some embodiments, the polymer is a polyvinylpyrrolidone. In other embodiments, the polymer is a triblock polyether polymer such as a poloxamer. In some embodiments, the polymer is a polysaccharide such as cellulose. In some embodiments, the polysaccharide is carboxymethyl cellulose. In other embodiments, the polysaccharide is a glucose polysaccharide. In some embodiments, the glucose polysaccharide is a mix of 1-4 and 1-6 glucose linkages. In some embodiments, the polysaccharide is dextrin. In other embodiments, the glucose polysaccharide is primarily 1-6 glucose linkages. In some embodiments, the polysaccharide is dextran.

In some embodiments, the pharmaceutical composition further comprises a protein. In some embodiments, the protein is a serum protein such as an albumin. In some embodiments, the protein is human serum albumin.

In some embodiments, the pharmaceutical compositions comprise from about 1% w/v to about 40% w/v of the sugar or sugar alcohol. In some embodiments, the pharmaceutical compositions comprise from about 2% w/v to about 30% w/v of the sugar or sugar alcohol. In some embodiments, the pharmaceutical compositions comprise from about 3% w/v to about 20% w/v of the sugar or sugar alcohol. In some embodiments, the pharmaceutical compositions comprise from about 7% w/v to about 16% w/v of the sugar or sugar alcohol. In some embodiments, the pharmaceutical compositions comprise about 7.5% w/v of the sugar or sugar alcohol. In some embodiments, the pharmaceutical compositions comprise about 15% w/v of the sugar or sugar alcohol.

In some embodiments, the pharmaceutical compositions comprise from about 0.01% w/v to about 5% w/v of the antioxidant. In some embodiments, the pharmaceutical compositions comprise from about 0.02% w/v to about 2.5% w/v of the antioxidant. In some embodiments, the pharmaceutical compositions comprise from about 0.05% w/v to about 2% w/v of the antioxidant. In some embodiments, the pharmaceutical compositions comprise from about 0.075% w/v to about 0.5% w/v of the antioxidant. In some embodiments, the pharmaceutical compositions comprise about 0.1% w/v of the antioxidant.

In some embodiments, the pharmaceutical compositions comprise from about 0.01% w/v to about 15% w/v of the polymer. In some embodiments, the pharmaceutical compositions comprise from about 0.1% w/v to about 10% w/v of the polymer. In some embodiments, the pharmaceutical compositions comprise from about 0.5% w/v to about 7.5% w/v of the polymer. In some embodiments, the pharmaceutical compositions comprise from about 0.75% w/v to about 5% w/v of the polymer. In some embodiments, the pharmaceutical compositions comprise about 1% w/v of the polymer. In some embodiments, the pharmaceutical compositions comprise from about 1% w/v to about 30% w/v of the polymer. In some embodiments, the pharmaceutical compositions comprise from about 1.5% w/v to about 20% w/v of the polymer. In some embodiments, the pharmaceutical compositions comprise from about 2% w/v to about 15% w/v of the polymer. In some embodiments, the pharmaceutical compositions comprise from about 2.5% w/v to about 12.5% w/v of the polymer. In some embodiments, the pharmaceutical compositions comprise about 5%, 7.5%, or 10% w/v of the polymer.

In some embodiments, the pharmaceutical compositions comprise from about 1% to about 35% w/v of the protein. In some embodiments, the pharmaceutical compositions comprise from about 2% to about 25% w/v of the protein. In some embodiments, the pharmaceutical compositions comprise from about 3% to about 20% w/v of the protein. In some embodiments, the pharmaceutical compositions comprise from about 4% to about 17.5% w/v of the protein. In some embodiments, the pharmaceutical compositions comprise 5%, 10%, or 15% w/v of the protein.

In some embodiments, the pharmaceutical compositions comprise an antioxidant and a polymer. In some embodiments, the pharmaceutical compositions comprise a first polymer and a second polymer. In some embodiments, the pharmaceutical compositions comprise an antioxidant, a polymer, and a protein.

In some embodiments, the pharmaceutical compositions comprise a buffer such as Dulbecco phosphate buffered saline. In some embodiments, the pharmaceutical composition comprises a cell culture medium such as Dulbecco Modified Eagle Medium (DMEM). In some embodiments, the pharmaceutical composition comprises an isotonic solution such as Plasma-Lyte.

In some embodiments, the pharmaceutical composition comprises:
(A) one or more eukaryotic cell;
(B) trehalose;
(C) epigallocatechin gallate; and
(D) polyvinylpyrrolidone.

In some embodiments, the pharmaceutical compositions further comprise an excipient. In some embodiments, the pharmaceutical compositions further comprise an amino acid. In some embodiments, the pharmaceutical compositions are formulated for administration intravenously. In other embodiments, the pharmaceutical compositions are formulated for administration via intraperitoneal injection, subcutaneous injection, or intratumoral injection. In other embodiments, the pharmaceutical compositions are formulated for administration via inhalation to the lungs. In other embodiments, the pharmaceutical compositions are formulated for administration topically to a surgically exposed site. In some embodiments, the pharmaceutical compositions are formulated for reconstitution in a solution.

In some embodiments, the pharmaceutical compositions show a decrease of less than about 10% change in viability compared to the pharmaceutical composition within 1 hour of preparation after storage at a temperature for 1 day. In some embodiments, the pharmaceutical compositions show a decrease of less than about 10% change in viability after storage at a temperature for 1 week. In some embodiments, the pharmaceutical compositions show a decrease of less than about 10% change in viability after storage at a temperature for 2 weeks. In some embodiments, the pharmaceutical compositions show a decrease of less than about 10% change in viability after storage at a temperature for 1 month. In some embodiments, the pharmaceutical compositions show a decrease of less than about 10% change in viability after storage at a temperature for 3 months. In some embodiments, the pharmaceutical compositions show a decrease of less than about 10% change in viability after storage at a temperature for 6 months. In some embodiments, the pharmaceutical compositions show a decrease of less than about 10% change in viability after storage at a temperature for 9 months. In some embodiments, the pharmaceutical compositions show a decrease of less than about 10% change in viability after storage at a temperature for 1 year. In some embodiments, the pharmaceutical compositions show a decrease of less than about 10% change in viability after storage at a temperature for 2 years. In some embodiments, the pharmaceutical compositions show a decrease of less than about 10% change in viability after storage at a temperature for 10 years. In some embodiments, the pharmaceutical compositions show a decrease of less than about 10% change in viability after storage at a temperature for 100 years.

In some embodiments, the change in activity is a decrease of less than about 7.5%. In some embodiments, the change in activity is a decrease of less than about 5%. In some embodiments, the change in activity is a decrease of less than about 2.5%. In some embodiments, the change in activity is a decrease of less than about 1%.

In some embodiments, the temperature is less than 25° C. In some embodiments, the temperature is from about −200° C. to about 25° C. In some embodiments, the temperature is from about −200° C. to about −160° C. In some embodiments, the temperature is from about −100° C. to about −60° C. In some embodiments, the temperature is from about −10° C. to about 15° C. In some embodiments, the temperature is from about 0° C. to about 10° C.

In some aspects, the present disclosure provides methods of preparing a pharmaceutical composition described herein, wherein the method comprises:
(A) incubating the eukaryotic cell with the sugar or sugar alcohol for an incubation time at an incubation temperature in a buffer, a cell culture medium, or an isotonic solution to form a pharmaceutical mixture;
(B) applying the pharmaceutical mixture to a frozen surface at a surface temperature below 0° C. to obtain a frozen pharmaceutical mixture; and
(C) collecting the frozen pharmaceutical mixture and drying the frozen pharmaceutical mixture to obtain a pharmaceutical composition.

In some embodiments, the buffer is Dulbecco phosphate buffered saline (DPBS). In some embodiments, the cell culture medium is Dulbecco Modified Eagle Medium (DMEM). In some embodiments, the isotonic solution is Plasma-Lyte.

In some embodiments, the incubation temperature is from about 10° C. to about 45° C. In some embodiments, the incubation temperature is from about 20° C. to about 40° C. In some embodiments, the incubation temperature is about 37° C. In some embodiments, the incubation is done under 5% Co₂.

In some embodiments, the incubation time is from about 15 minutes to about 24 hours. In some embodiments, the incubation time is from about 30 minutes to about 18 hours. In some embodiments, the incubation time is from about 1 hours to about 12 hours. In some embodiments, the incubation time is from about 3 hours to about 6 hours.

In some embodiments, the methods further comprise admixing the antioxidant or the polymer to the pharmaceutical mixture after incubation. In some embodiments, the methods further comprise incubating the antioxidant or the polymer with the eukaryotic cells. In some embodiments, the methods further comprise changing the incubation solution to a second incubation solution with a reduced concentration of the sugar or sugar alcohol. In some embodiments, the pharmaceutical mixture is applied at a feed rate from about 0.5 mL/min to about 5 m/min. In some embodiments, the feed rate is from about 1 mL/min to about 3 m/min. In some embodiments, the feed rate is about 2 mL/min. In some embodiments, the pharmaceutical mixture is applied with a nozzle such as a large bore needle or a pipette tip.

In some embodiments, the pharmaceutical mixture is applied from a height from about 0.25 cm to about 10 cm above the frozen surface. In some embodiments, the height is from about 0.5 cm to about 5 cm such as about 1 cm. In some embodiments, the surface temperature is from about 0° C. to −190° C. In some embodiments, the surface temperature is from about −25° C. to about −125° C. such as about −80° C.

In some embodiments, the frozen surface is a stationary surface. In some embodiments, the frozen surface is a rotating surface on a cryogenically cooled drum. In some embodiments, the surface is rotating at a speed from about 5 rpm to about 500 rpm. In some embodiments, the surface is rotating at a speed from about 100 rpm to about 400 rpm such as about 200 rpm.

In some embodiments, the pharmaceutical mixture is applied to the surface as droplets with a diameter from about 0.5 mm to about 10 mm. In some embodiments, the diameter is from about 1 mm to about 5 mm. In some embodiments, the diameter is from about 1.5 mm to about 2.5 mm.

In some embodiments, the methods produce a thin film with a diameter from about 0.5 mm to about 25 mm. In some embodiments, the thin film diameter is from about 2 mm to about 20 mm. In some embodiments, the thin film diameter is from about 3 mm to about 15 mm. In some embodiments, the thin film diameter is from about 4 mm to about 10 mm. In some embodiments, the thin film has a thickness from about 0.01 mm to about 15 mm. In some embodiments, the thin film has a thickness from about 0.05 mm to about 10 mm. In some embodiments, the thin film has a thickness from about 0.075 mm to about 7.5 mm. In some embodiments, the thin film has a thickness from about 0.1 mm to about 5 mm.

In some embodiments, the frozen pharmaceutical composition is dried by lyophilization. In some embodiments, the frozen pharmaceutical composition is dried at a reduced pressure. In some embodiments, the reduced pressure is from about 10 mTorr to 500 mTorr. In some embodiments, the reduced pressure is from about 50 mTorr to about 250 mTorr such as about 100 mTorr.

In some embodiments, the frozen pharmaceutical composition is dried at a reduced temperature. In some embodiments, the reduced temperature is from about 37° C. to −100° C. In some embodiments, the reduced temperature is from about −20° C. to about −60° C. such as about −35° C. In some embodiments, the frozen pharmaceutical composition is dried for a primary drying time period from about 3 hours to about 36 hours. In some embodiments, the primary drying time period is from about 6 hours to about 24 hours. In some embodiments, the primary drying time period is about 12 hours.

In some embodiments, the methods comprise a secondary drying period. In some embodiments, the frozen pharmaceutical composition is dried by a secondary lyophilization. In some embodiments, the frozen pharmaceutical composition is dried at a second reduced pressure. In some embodiments, the second reduced pressure is from about 10 mTorr to 500 mTorr. In some embodiments, the second reduced pressure is from about 50 mTorr to about 250 mTorr such as about 100 mTorr.

In some embodiments, the frozen pharmaceutical composition is dried at a second reduced temperature. In some embodiments, the second reduced temperature is from about 37° C. to −100° C. In some embodiments, the second reduced temperature is from about −20° C. to about −60° C. such as about −35° C. In some embodiments, the frozen pharmaceutical composition is dried for a secondary drying time period from about 3 hours to about 36 hours. In some embodiments, the secondary drying time period is from about 6 hours to about 24 hours such as about 12 hours.

In still yet another aspect, the present disclosure provides pharmaceutical compositions prepared using the methods described herein.

In still another aspect, the present disclosure provides methods of treating or preventing a disease or disorder comprising administering to a patient in need thereof a therapeutically effective amount of a pharmaceutical composition described herein.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1G show the effect of trehalose concentration and incubation time on J774A.1 cell viability after thin-film freeze-drying (TFFD). Data are mean±S.D. (n=3). Cells were incubated in trehalose solutions of various concentrations (0-30%, w/v) for various periods of time (0-18 h) before subjected to TFFD. Cells need to be incubated with trehalose for at least 2 h before they are subjected to TFFD for trehalose to protect the cells. The optimum condition is to incubate the cells with trehalose at 15% w/v (or 0.4 mM) for 6 h in a CO₂ incubator at 37° C.

FIGS. 2A-2C show the effect of EGCG concentration and incubation time on J774A.1 cell viability after TFFD. Data are mean±S.D. (n=3). The optimum concentration of EGCG to help maintain maximum cell viability after TFFD was 0.1% w/v. Also, 6 h of incubation of the cells with EGCG was enough.

FIGS. 3A-3E show the effect of D-α-tocopherol succinate, D-α-tocopherol PEG-1000 succinate, ascorbic acid, glutathione, and trolox as antioxidants on J774A.1 cell viability after TFFD. Data are mean±S.D. (n=3). Trolox at 500 M and D-α-tocopherol succinate at 800 μg/ml helped improve the viability of the cells after subjected to TFFD.

FIGS. 4A-4E show the effect of different polymers P-188, PVP-40, dextran, CMC, and dextrin and their concentration on J774A.1 cell viability after TFFD. Data are mean±S.D. (n=3). Polyvinylpyrrolidone-40 (PVP-40), P-188, and dextrin showed a positive, concentration-dependent effect on cell viability after TFFD.

FIG. 5 shows the effect of the mixture of trehalose (7.5%) with EGCG (1 mg/ml), PVP-40 (20%), and/or P-188 (20%) on J774A.1 cell viability after TFFD. Data are mean±S.D. (n=3). Trehalose with PVP-40 or P188, with or without EGCG, were effective in maintaining cell viability.

FIGS. 6A & 6B show the stability of thin-film freeze-dried J774A.1 cells when stored at 4° C. (A) Dry powders of J774A.1 cells prepared with trehalose (7.5%) with EGCG (1 mg/ml), PVP-40 (20%), and/or P-188 (20%) after 50 days of storage at 4° C. (B) Dry powders of J774A.1 cells prepared with trehalose (15%), EGCG or Trolox, and PVP-40 or P-188 after 15 and 30 days of storage at 4° C. Please see Table 1 for details of the compositions. Data are mean±S.D. (n=3).

FIG. 7 shows the effect of the distance between the pipette tip and the frozen surface on the diameter of the resulting frozen thin-films. THP-1 cells (200 μl, 1.0×10⁶ cells/mL in a 10 mM DPBS (pH 7.4) with 7.5% trehalose, 0.1% EGCG, and 1% PVP-40, all w/v) were dropped from a distance of 1 cm, 2 cm, 5 cm, or 10 cm above the frozen surface in 7-8 drops and the diameter of the frozen thin-films was estimated using a caliper. At 10 cm, the droplets splashed, resulting in smaller pieces of frozen thin-films with irregular shapes. Cells were preincubated with trehalose and EGCG at 37° C. in a CO₂ incubator before subjected to thin-film freezing drying (TFFD), while PVP40 was simply added before the TFFD.

FIG. 8 shows the B16-F10 viability after subjected to TFFD using an MTT assay. Shown are numbers of live B16-F10 cells after thin-film freeze-dried B16-F10 cells were reconstituted and incubated in DMEM media for different time intervals. Cells cryopreserved in 5% DMSO were the positive control, while cells subjected to TFFD without any excipient were the negative control. Data (n=3) were based on an MTT assay using a calibration curve showing the relationship between the numbers of B16-F10 cells and their corresponding optical density values at 550 nm.

### DESCRIPTION OF ILLUSTRATIVE EMBODIMENTS

The present disclosure provides dry powder compositions of eukaryotic cells that do not contain DMSO and can be stored and shipped in refrigerated conditions (e.g., 2-8° C.). These dry powders were prepared by thin-film freeze-drying (TFFD). Thin-film freezing (TFF) technique is an ultra-rapid freezing technology that has a freeze rate of 100-1000 Kelvin per second, without the large air-liquid interface associated with other ultra-rapid freezing methods. Cells are suspended in an aqueous solution free of DMSO and spread out on a cryogenically cooled surface to rapidly form frozen thin-films. Water molecules may then be removed from the frozen thin-films by sublimation to form brittle matrix powders with low density and high surface area, allowing rapid reconstitution. The viability of the cells is preserved in these thin-films after the eukaryotic cells are subjected to TFFD, and the resultant cell dry powders can be stored in refrigerated temperatures for an extended period of time.

In one embodiment, murine or human monocytes were subjected to TFFD and exhibited as high as 99% of the cells in the resultant dry powders retaining viable upon reconstitution as measured by Trypan Blue staining. Additionally, after more than seven weeks of storage of the thin-film freeze-dried cell powders in a refrigerator (4° C.), the viability of the cells remained unchanged. These and other benefits of the present compositions will be described herein.

### I. Ultra-Rapid Freezing (URF) Formulation

In certain aspects, the present disclosure provides pharmaceutical compositions which may be prepared using a URF process, such as thin-film freezing process. Methods of preparing pharmaceutical compositions using thin film freezing are described in U.S. Patent Application No. 2010/0221343, Watts, et al., 2013, Engstrom et al. 2008, Wang et al. 2014, Thakkar at el. 2017, O'Donnell et al. 2013, Lang et al. 2014a, Lang et al. 2014b, Carvalho et al. 2014, Beinborn et al. 2012a, Beinborn et al. 2012b, Zhang et al. 2012, Overhoff et al. 2009, Overhoff et al. 2008, Overhoff et al. 2007a, Overhoff et al. 2007b, Watts et al. 2010, Yang et al. 2010, DiNunzio et al. 2008, Purvis et al. 2007, Liu et al. 2015, Sinswat et al. 2008, and U.S. Pat. No. 8,968,786, all of which are incorporated herein by reference. In some cases, the methods employ an ultra-rapid freezing rate of up to 10,000 K/sec, e.g., at least 1,000, 2,000, 5,000 or 8,000 K/sec. In some embodiments, these methods involve dissolving the components of the pharmaceutical composition into a solvent, such as water or a buffer, to form a pharmaceutical mixture. The solvent may be water. In other embodiments, the solvent may be a buffer. However, the in preferred aspects, the pharmaceutical mixture is an aqueous solution that includes eukaryotic cells, a sugar or sugar alcohol, or an antioxidant or a polymer. In some embodiments, the pharmaceutical mixture may contain less than 10% w/v of the eukaryotic cells, the sugar or sugar alcohol, or the antioxidant or the polymer. The pharmaceutical mixture may contain less than 0.5%, 1%, 2%, 3%, 4%, 5%, 6%, 7%, 8%, 9%, or 10% w/v, or any range derivable therein.

In some aspects, the eukaryotic cells are incubated with the sugar or sugar alcohol at an incubation temperature. The incubation temperature may be about 20° C., 25° C., 27.5° C., 30° C., 32.5° C., 35° C., 36° C., 38° C., 40° C., 42° C., 44° C., 45° C., 46° C., 48° C., or 50° C. The incubation may occur under ambient conditions or may occur under a CO₂ atmosphere. The incubation may occur for an incubation time from about 15 minutes, 30 minutes, 45 minutes, 1 hour, 1.5 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 1.5 days, or 2 days, or any range derivable therein.

This pharmaceutical mixture may be deposited on a surface which is at a temperature that causes the pharmaceutical mixture to freeze. In some embodiments, this temperature may be below the freezing point of the solution at ambient pressure. In other embodiments, a reduced pressure may be applied to the surface causing the solution to freeze at a temperature below the ambient pressure's freezing point. The surface may also be rotating or moving on a moving conveyer-type system thus allowing the pharmaceutical mixture to distribute evenly on the surface. In another aspect, the surface may be stationary. Alternatively, the pharmaceutical mixture may be applied to surface in such a manner to generate an even surface. The pharmaceutical mixture may be applied at a feed rate from about 0.5 mL/min to about 5 m/min, 1 mL/min to about 3 mL/min, or from about 0.25 mL/min, 0.5 mL/min, 0.75 mL/min, 1 mL/min, 1.5 m/min, 2 m/min, 2.5 mL/min, 3 mL/min, 4 m/min, to about 5 mL/min, or any range derivable therein. In some embodiments, the pharmaceutical mixture is applied to the surface using a nozzle such as a large bore needle or a pipette tip. The pharmaceutical mixture is applied from a height from about 0.25 cm to about 10 cm above the frozen surface, 0.5 cm to about 5 cm, or from about 0.75 cm to about 2 cm. The height may be from about 0.1 cm, 0.25 cm, 0.5 cm, 0.75 cm, 1 cm, 1.25 cm, 1.5 cm, 1.75 cm, 2 cm, 2.5 cm, 3 cm, 3.5 cm, 4 cm, to about 5 cm, or any range derivable therein. Furthermore, the surface may have a surface temperature of less than 10° C., less than 0° C., or less than −20° C. The surface temperature may be from about 0° C. to −190° C. or from about −25° C. to about −125° C. The surface temperature may be from about 10° C., 0° C., −10° C., −20° C., −30° C., −40° C., −50° C., −60° C., −70° C., −80° C., −90° C., −100° C., −120° C., −140° C., −160° C., −180° C., −190° C., or −200° C., or any range derivable therein.

After the pharmaceutical mixture has been applied to the surface to form frozen thin-films, the solvent may be removed to obtain a pharmaceutical composition. Any appropriate method of removing the solvent may be applied including evaporation under reduced pressure or elevated temperature or lyophilization. In some embodiments, the lyophilization may comprise a reduced pressure and/or a reduced temperature. Such a reduced temperature may be from 25° C. to about −200° C., from 20° C. to about −175° C., from about 20° C. to about −150° C., from 0° C. to about −125° C., from −20° C. to about −100° C., from −75° C. to about −175° C., or from −100° C. to about −160° C. The temperature is from about −20° C., −30° C., −35° C., −40° C., −45° C., −50° C., −55° C., −60° C., −70° C., −80° C., −90° C., −100° C., −110° C., −120° C., −130° C., −140° C., −150° C., −160° C., −170° C., −180° C., −190° C., to about −200° C., or any range derivable therein. Additionally, the solvent may be removed at a reduced pressure of less than 500 mTorr, 450 mTorr, 400 mTorr, 375 mTorr, 350 mTorr, 325 mTorr, 300 mTorr, 275 mTorr, 250 mTorr, 225 mTorr, 200 mTorr, 175 mTorr, 150 mTorr, 125 mTorr, 100 mTorr, 75 mTorr, 50 mTorr, or 25 mTorr.

Such a composition prepared using these methods may exhibit a brittle nature such that the composition is easily sheared into smaller particles when processed through a device. These compositions have high surface areas as well as exhibit improved flowability of the composition. Such flowability may be measured, for example, by the Carr's index or other similar measurements. In particular, the Carr's index may be measured by comparing the bulk density of the powder with the tapped density of the powder. Such composition may exhibit a favorable Carr's index and may result in the particles being better sheared to give smaller particles when the composition is processed through a secondary device to further process a powder composition.

### II. Intact Cells

In some aspects, compositions of the embodiments comprise intact and/or living cells. For example, the cells can be eukaryotic cells. For example, the cells can comprise human cells (e.g., human iPS cells), fungal cells (e.g., yeast cell), or plant cells. In some embodiments, the cells may be genetically engineered cells or contain an altered genetic code such that the eukaryotic cells produce a unique protein such as CAR-T cells. In other embodiments, the cells may be stem cells that are used to generate a new tissue. In some embodiments, the cells may be cell lines. Yet in other embodiments, the cells may be primary cells isolated from a subject such as a patient.

### III. Excipients

### A. Sugars or Sugar Alcohols

In some aspects, the present disclosure comprises one or more excipients formulated into pharmaceutical compositions. In some embodiments, the excipients used herein are water soluble excipients. These water-soluble excipients include sugar or sugar alcohol. The sugar or sugar alcohol may be a saccharide such as disaccharide. In some cases, the excipient comprises sucrose, trehalose, or lactose, a trisaccharide such as fructose, sucrose, glucose, galacatose, or raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol.

In some embodiments, the excipient, especially the sugar or sugar alcohol, comprises from about 1% w/v to about 35% w/v, from about 2% w/v to about 25% w/v, from about 3% w/v to about 20% w/v, or from about 7% w/v to about 16% w/v of the sugar or sugar alcohol. The amount of the sugar or sugar alcohol may be from about 1% w/v, 2% w/v, 3% w/v, 4% w/v, 5% w/v, 6% w/v, 7% w/v, 8% w/v, 9% w/v, 10% w/v, 11% w/v, 12% w/v, 13% w/v, 14% w/v, 15% w/v, 16% w/v, 17% w/v, 18% w/v, 20% w/v, 22% w/v, 24% w/v, 25% w/v, 26% w/v, 28% w/v, 30% w/v, 32.5% w/v, 35% w/v, 37.5% w/v, to about 40% w/v, or any range derivable therein.

### B. Antioxidants

In another aspect, the present disclosure may further comprise one or more antioxidants. Antioxidant compositions used in the pharmaceutical compositions may comprise single antioxidants or combinations of two or more antioxidants.

The antioxidants used may include natural exogenous phytochemical antioxidants such as phenolics and carotenoids. In some embodiments, the antioxidants used may include flavonoids. Flavonoids constitute a large group of over 5000 polyphenolic phytochemicals with antioxidant properties that act through direct free radicals scavenging. Flavonoids have anti-inflammatory, anti-bacterial, anti-viral, anti-allergic, anti-mutagenic, anti-thrombotic, anti-neoplastic and vasodilatory action and may prevent, reduce, or eliminate the oxidative damage from dental devices using these methods of action as well. Flavonoids also exhibit chelating properties with metal ions and may reduce the oxidative damage from metal ions by sequestering the ions. Formation and stability of flavonoids-metal-chelates is a structure-dependent function. Flavonoids with a catechol moiety and with hydrogen bonds between hydroxyl group in the 5- and 3-positions have chelating properties. Glycosides of flavonoids may also be used.

In some embodiments, the flavonoids may be a flavanone (derivative of 2,3-dihydro-2-phenylchromen-4-one). Flavones include Butin, Eriodictyol, Hesperetin, Hesperidin, Homoeriodictyol, Isosakuranetin, Naringenin, Naringin, Pinocembrin, Poncirin, Sakuranetin, Sakuranin, and Sterubin.

In other embodiments, the flavonoid may be a flavanonol (derivative of 3-hydroxy-2,3-dihydro-2-phenylchromen-4-one). Flavanols include Taxifolin, Aromadedrin, Chrysandroside A, Chrysandroside B, Xeractinol, Astilbin, and Fustin.

In other embodiments, the flavonoid may be a flavone (derivative of 2-phenylchromen-4-one). Flavones include: Apigenin, Luteolin, Tangeritin, Chrysin, Baicalein, Scutellarein, Wogonin, Synthetic Flavones: Diosmin, and Flavoxate.

In other embodiments, the flavonoid may be a flavonol (derivative of 3-hydroxy-2-phenylchromen-4-one). Flavonols include: 3-Hydroxyflavone, Azaleatin, Fisetin, Galangin, Gossypetin, Kaempferide, Kaempferol, Isorhamnetin, Morin, Myricetin, Natsudaidain, Pachypodol, Quercetin, Rhamnazin, Rhamnetin, Azalein, Hyperoside, Isoquercitin, Kaempferitrin, Myricitrin, Quercitrin, Robinin, Rutin, Spiraeoside, Xanthorhamnin, Amurensin, Icariin, and Troxerutin.

In other embodiments, the flavonoid may be a flavan-3-ol (derivatives of 2-phenyl-3,4-dihydro-2H-chromen-3-ol). Flavan-3-ols include: Catechin, Epicatechin, Epigallocatechin, Epicatechin gallate, Epigallocatechin gallate, Epiafzelechin, Fisetinidol, Guibourtinidol, Mesquitol, and Robinetinidol.

In other embodiments, the flavonoid may be a flavan-4-ol (derivative of 2-phenylchroman-4-ol). Flavan-4-ols include: Apiforol and Luteoforol. In still another embodiment, the flavonoid may be an isoflavone (derivative of 3-phenylchromen-4-one). Isoflavones include: Genistein, Daidzein, Biochanin A, Formononetin, and the Equol metabolite from Daidzein.

In other embodiments, the antioxidant may be an anthocyanidin (derivative of 2-phenylchromenylium cation). Anthocyanidins include: Aurantinidin, Cyanidin, Delphinidin, Europinidin, Luteolinidin, Pelargonidin, Malvidin, Peonidin, Petunidin, Rosinidin, and Xanthone.

In other embodiments, the antioxidant may be a dihydrochalcone (derivative of 1,3-diphenyl-1-propanone). Dihydrochalcones include: Phloretin, Dihydrochalcone phloretin Phlorizin, Aspalathin, Naringin dihydrochalcone, Neohesperidin dihydrochalcone, and Nothofagin. Without limiting the mode of action of the invention, dihydrochalcones may exert an antioxidant effect by reducing reactive free radicals, like reactive oxygen and reactive nitrogen species.

In other embodiments, the antioxidant may be a Phenylpropanoid (derivatives of cinnamic acid). Phenylpropanoids include: Cinnamic acid, Caffeic acid, Ferulic acid, Trans-ferulic acid (including its antioxidant pharmacore 2,6-dihydroxyacetophenome), 5-Hydroxyferulic acid, Sinapic acid, Coumaryl alcohol, Coniferyl alcohol, Sinapyl alcohol, Eugenol, Chavicol, Safrole, P-coumaric acid, and Sinapinic acid. Without limiting the mode of action of the invention, Phenylpropanoids may neutralize free radicals.

In other embodiments, the antioxidant may be a Chalcone (derivative of 1,3-diphenyl-2-propen-1-one). Chalcones include: Butein, Okanin, Carthamin, Marein, Sophoradin, Xanthohumol, Flavokvain A, Flavokavain B, Flavokavin C, and synthetic Safalcone.

In other embodiments, the antioxidant may be a Curcuminoid.

Curcuminoids include: Curcumin, Desmethoxycurcumin, bis-Desmethoxycurcumin, Tetrahydrocurcumin, and Tetrahydrocurcuminoids. Curcumin and tetrahydrocurcuminoids may be derived from rhizomes of Curcuma longa. Tetrahydrocurcumin, a metabolite of curcumin, has been found to be a more potent antioxidant and more stable compared to curcumin. Tetrahydrocurcumin is available commercially, for example, it is the main component of Tetrahydrocurcuminoids CG™ as sold by Sabinsa Corp. (Piscataway, N.J.). Tetrahydrocurcuminoids CG™ contains on a w/w basis tetrahydrocurcumin (75-90%), tetrahydrodemethoxycurcumin (15-20%), and tetrahydrobisdemethoxycurcumin (1-4%). Each of these components is a potent antioxidant. Accordingly, in some embodiments, curcumin or tetrahydrocurcumin may be used in place of tetrahydrocurcuminoids. Further, each component of Tetrahydrocurcuminoids CG™ may be used separately as tetrahydrocurcuminoids. Tetrahydrocurcuminoids CG™ or other useful tetrahydrocurcuminoids are described in WO 00/61162.

In other embodiments, the antioxidant may be a Tannin. Tannins include: Tannin, Terflavin B, Glucogallin, gallic acid, and Quercitannic acid.

In other embodiments, the antioxidant may be a stilbenoid. Stilbenoids include: Resveratrol, Pterostilbene, and Piceatannol. Resveratrol may include, but is not limited to, 3,5,4′-trihydroxystilbene, 3,4,3′,5′-tetrahydroxystilbene (piceatannol), 2,3′,4,5′-tetrahydroxystilbene (oxyresveratrol), 4,4′-dihydroxystilbene, and alpha and beta glucoside, galactoside and mannoside derivatives thereof. Other derivatives are recited in U.S. Pat. No. 6,572,882, incorporated by reference herein. Additionally, analogs of resveratrol such as the 3,4,4′,5-tetrahydroxystilbene of U.S. Pat. No. 6,790,869, incorporated by reference herein, may also be used. Both cis and trans configurations of resveratrol or its derivatives may be used. Without limiting the mode of action of the invention, stilbenoids may neutralize free radicals.

In other embodiments, the antioxidant may be a Coumarin (derivatives of 2H-chromen-2-one). Coumarins include: 4-Hydroxycoumarin, Umbelliferone, Aesculetin, Herniarin, Auraptene, and Dicoumarol.

In other embodiments, the antioxidant may be a Carotenoid.

Carotenoids include: beta-Carotene, alpha-Carotene, gamma-Carotene, beta-Cryptoxanthin, Lycopene, Lutein, and Idebenone.

In other embodiments, the antioxidant may be a vitamin. Vitamins include: Retinol, Ascorbic acid, L-Ascorbic acid, Tocopherol, Tocotrienol, and the Vitamin cofactor: Coenzyme Q10.

In some aspects, the present disclosure provides a vitamin or vitamin derivative which includes a polyethylene glycol (PEG) polymer with a molecular weight from about 100 to about 4000 daltons, from about 100 to about 1000 daltons, from about 100 to about 500 daltons, or from about 100, 200, 300, 400, 500, 600, 700, 800, 900, 1000, 1250, 1500, 1750, 2000, 2500, 3000, 3500, or about 4000 daltons. In some embodiments, the PEG polymer further comprises a hydrophobic group such as a vitamin or fatty acid. In some embodiments, the hydrophobic group may be a vitamin such as vitamin E. Such a compound may further comprise a linking group such as a diamine or dicarboxylic acid such as 1,2-ethylenediamine or succinic acid. The vitamin or vitamin derivative group may be a PEGylated tocopherol succinate such as TPGS 1000 or similar tocopherol succinate compounds.

In other embodiments, the antioxidant may be: a Xanthone, Butylated Hydroxytoluene, 2,6-Di-tert-butylphenol, 2,4-Dimethyl-6-tert-butylphenol, Gallic acid, Eugenol, Uric acid, alpha-Lipoic acid, Ellagic acid, Chicoric acid, Chlorogenic acid, Rosmarinic acid, Salicylic acid, Acetylcysteine, S-Allyl cysteine, Barbigerone, Chebulagic acid, Edaravone, Ethoxyquin, Glutathione, Hydoxytyrosol, Idebenone, Melatonin, N-Acetylserotonin, Nordihydroguaiaretic acid, Oleocanthal, Oleuropein, Paradol, Piceatannol, Probucol, Propyl gallate, Protocatechuic acid, Pyritinol, Rutin, Secoisolariciresinol diglucoside, Sesamin, Sesamol, Silibinin, Silymarin, Theaflavin, Theaflavin digallate, Thmoquinone, Trolox, Tyrosol, Polyunsaturated fatty acids, and sulfur-based antioxidants such as Methionine or Lipoic acid.

Antioxidants used herein may be synthesized, extracted or purified from natural products, or present in a natural product. The antioxidants may be isolated or partially isolated prior to formulation for use in the methods described herein or used in a naturally occurring form. In addition, the antioxidants may include plant extracts or combinations containing any of the above mentioned antioxidants or derivatives thereof.

In some aspects, the amount of the antioxidant in the pharmaceutical compositions is from about 0.01% w/v to about 10% w/v, from about 0.1% w/v to about 7.5% w/v, from about 0.5% w/v to about 5% w/v, from about 0.75% w/v to about 4% w/v, from about 1% w/v to about 25% w/v, from about 1.5% w/v to about 20% w/v, from about 2% w/v to about 15% w/v, or from about 2.5% w/v to about 12.5% w/v. The amount of the antioxidant in the pharmaceutical composition comprises from about 0.01%, 0.02%, 0.025%, 0.05%, 0.075%, 0.1%, 0.2%, 0.25%, 0.3%, 0.4%, 0.5%, 0.75%, 1%, 1.25%, 1.5%, 1.75%, 2%, 2.5%, 3%, 3.5%, 4%, 4.5%, 5%, 6%, 7%, 8%, 9%, 10%, 11%, 12%, 13%, 14%, 15%, 16%, 18%, 20%, 22%, 24%, 25%, to about 30% w/v, or any range derivable therein.

### C. Polymers

Within the compositions described herein, a single polymer or a combination of multiple polymers may be used. In some embodiments, the polymers used herein may fall within two classes: cellulosic and non-cellulosic. These classes may be further defined by their respective charge into neutral and ionizable. Ionizable polymers have been functionalized with one or more groups which are charged at a physiologically relevant pH. Some non-limiting examples of neutral non-cellulosic polymers include polyvinyl pyrrolidone, polyvinyl alcohol, copovidone, and poloxamer. Within this class, in some embodiments, pyrrolidone containing polymers are particularly useful. Some non-limiting examples of ionizable cellulosic polymers include cellulose acetate phthalate and hydroxypropyl methyl cellulose acetate succinate. Finally, some non-limiting examples of neutral cellulosic polymers include hydroxypropyl cellulose, hydroxypropyl methyl cellulose, hydroxyethyl cellulose, and hydroxymethyl cellulose.

Some specific pharmaceutically acceptable polymers which may be used include, for example, Eudragit™ RS PO, Eudragit™ S100, Kollidon SR (poly(vinyl acetate)-co-poly(vinylpyrrolidone) copolymer), Ethocel™ (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PVA), hydroxypropyl methylcellulose (HPMC), ethylcellulose (EC), hydroxyethylcellulose (HEC), carboxymethyl cellulose and alkali metal salts thereof, such as sodium salts sodium carboxymethyl-cellulose (CMC), dimethylaminoethyl methacrylate—methacrylic acid ester copolymer, carboxymethylethyl cellulose, carboxymethyl cellulose butyrate, carboxymethyl cellulose propionate, carboxymethyl cellulose acetate butyrate, carboxymethyl cellulose acetate propionateethylacrylate—methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, poly(methacylic acid-co-methyl methacrylate 1:2), poly(methacrylic acid-co-methyl methacrylate 1:1), Poly(methyl acrylate-co-methyl methacrylate-co-methacrylic acid 7:3:1), poly(butyl methacrylate-co-(2-dimethylaminoethyl) methacrylate-co-methyl methacrylate 1:2:1), poly(ethyl acrylate-co-methyl methacrylate 2:1), poly(ethyl acrylate-co-methyl methacrylate 2:1), poly(ethyl acrylate-co-methyl methacrylate-co-trimethylammonioethyl methacrylate chloride 1:2:0.2), poly(ethyl acrylate-co-methyl methacrylate-co-trimethylammonioethyl methacrylate chloride 1:2:0.1), Eudragit L-30-D™ (MA-EA, 1:1), Eudragit L-100-55™ (MA-EA, 1:1), hydroxypropylmethylcellulose acetate succinate (HPMCAS), polyvinyl caprolactam-polyvinyl acetate-PEG graft copolymer, polyvinyl alcohol/acrylic acid/methyl methacrylate copolymer, polyalkylene oxide, Coateric™ (PVAP), Aquateric™ (CAP), and AQUACOAT™ (HPMCAS), polycaprolactone, starches, pectins, chitosan or chitin and copolymers and mixtures thereof, and polysaccharides such as tragacanth, gum arabic, guar gum, and xanthan gum.

In some embodiments, the compositions described herein contain a pharmaceutically acceptable polymer selected from povidone, copovidone, polyvinyl pyrrolidone, polyvinyl acetate, and SOLUPLUS® (polyvinyl caprolactampolyvinyl acetate-polyethylene glycol graft co-polymer, commercially available from BASF). In particular, the pharmaceutical acceptable polymer may be a copolymer of polyvinyl pyrrolidone and polyvinyl acetate. In particular, the copolymer may comprise about 5-7 vinyl pyrrolidone units to about 3-5 units of vinyl acetate, in particular 6 units of vinyl pyrrolidone and 4 units of vinyl acetate. The number-average of the molecular weight of the polymer may be from about 15,000 to about 20,000. The pharmaceutically acceptable polymer may be Kollidan® VA 64 (copovidone, vinylpyrrolidone-vinyl acetate) having a CAS Number of 25086-89-9.

In some examples of the present disclosure comprises a polymer including PEG alkyl ethers, polypropylene glycol ethers, glucoside alkyl ethers, PEG alkylaryl ethers such as Triton® (2-[4-(2,4,4-trimethylpentan-2-yl)phenoxy]ethanol) and nonoxynol, simple alkyl esters of glycerol such as glycerol laurate, polysorbates such as Tween® (polyethylene glycol sorbitan monolaurate), Sorbitan alkyl esters such as Span, or Poloxamer® (triblock copolymers of polyethylene glycol and polypropylene glycol) and other block copolymers of polyethylene glycol and polypropylene glycol. In some embodiments, the polymers that may be used in the present pharmaceutical compositions contain one or more polyethylene glycol or polypropylene glycol polymer such as Tween® (polyethylene glycol sorbitan monolaurate), Capryol® (propylene glycol monocaprylate), Labrafil® (2-[2,3-bis(2-hydroxyethoxy) propoxy]ethanol; hexadecanoic acid; octadecanoic acid), or Labrasol® (caprylocaproyl macrogol-8 glycerides, caprylocaproyl polyoxyl-8 glycerides, polyoxylglycerides).

In some aspects, the amount of the polymer in the pharmaceutical compositions is from about 0.01% w/v to about 10% w/v, from about 0.1% w/v to about 7.5% w/v, from about 0.5% w/v to about 5% w/v, from about 0.75% w/v to about 4% w/v, from about 1% w/v to about 25% w/v, from about 1.5% w/v to about 20% w/v, from about 2% w/v to about 15% w/v, or from about 2.5% w/v to about 12.5% w/v. The amount of the polymer in the pharmaceutical composition comprises from about 0.01%, 0.025%, 0.05%, 0.1%, 0.2%, 0.25%, 0.3%, 0.4%, 0.5%, 0.75%, 1%, 1.25%, 1.5%, 1.75%, 2%, 2.5%, 3%, 3.5%, 4%, 4.5%, 5%, 6%, 7%, 8%, 9%, 10%, 11%, 12%, 13%, 14%, 15%, 16%, 18%, 20%, 22%, 24%, 25%, to about 30% w/v, or any range derivable therein.

### D. Proteins

In The pharmaceutical compositions described herein comprise protein such as a protein which is positively charged or negatively charged at physiological pH. This particular protein may be an endogenous protein. The protein may function to change the activity of the cells. In some aspects, the amount of the protein in the pharmaceutical compositions is from about 0.01% w/v to about 10% w/v, from about 0.1% w/v to about 7.5% w/v, from about 0.5% w/v to about 5% w/v, from about 0.75% w/v to about 4% w/v, from about 1% w/v to about 25% w/v, from about 1.5% w/v to about 20% w/v, from about 2% w/v to about 15% w/v, or from about 2.5% w/v to about 12.5% w/v. The amount of the protein in the pharmaceutical composition comprises from about 0.01%, 0.025%, 0.05%, 0.1%, 0.2%, 0.25%, 0.3%, 0.4%, 0.5%, 0.75%, 1%, 1.25%, 1.5%, 1.75%, 2%, 2.5%, 3%, 3.5%, 4%, 4.5%, 5%, 6%, 7%, 8%, 9%, 10%, 11%, 12%, 13%, 14%, 15%, 16%, 18%, 20%, 22%, 24%, 25%, to about 30% w/v, or any range derivable therein.

In some aspects, a wide variety of different forms of proteins may be contemplated to be used in the formulations described herein. In particular, these proteins may be ones that have been humanized or a human protein. The amino acid sequence of the protein may have also been modified in such a way that it reduces the degradation of the protein or immunogenicity. These modifications may alter the protein from being degraded during formulation, during storage, or in vivo. In another aspect, the present pharmaceutical composition may further comprise an endogenous protein that does not require modification to reduce degradation or immunogenicity.

### E. Other Excipients

In some aspects, the present pharmaceutical compositions may further include a hydrophobic or waxy excipient such as waxes and oils. In other aspects, the present pharmaceutical compositions may further exclude a hydrophobic or waxy excipient such as waxes and oils. Some non-limiting examples of hydrophobic excipients include hydrogenated oils and partially hydrogenated oils, palm oil, soybean oil, castor oil, carnauba wax, beeswax, palm wax, white wax, castor wax, or lanoline. The present disclosure is substantially free of any solvents such as dimethyl sulfoxide. In some embodiments, the pharmaceutical composition is more substantially free of any solvents such as dimethyl sulfoxide. In some embodiments, the pharmaceutical composition is essentially free of any solvents such as dimethyl sulfoxide.

Additionally, the present disclosure may further comprise one or more amino acids or an amide or ester derivative thereof. In some embodiments, the amino acids used may be one of the 20 canonical amino acids such as glycine, alanine, valine, isoleucine, leucine, methionine, phenylalanine, tyrosine, tryptophan, serine, threonine, asparagine, glutamine, cysteine, selenocysteine, proline, arginine, histidine, lysine, aspartic acid, or glutamic acid. These amino acids may be in the D or L orientation or the amino acids may be an α-, β-, γ-, or δ-amino acids. In other embodiments, one of the common non-canonical amino acids may be used such as carnitine, GABA, carboxyglutamic acid, levothyroxine, hydroxyproline, seleonmethionine, beta alanine, ornithine, citrulline, dehydroalanine, δ-aminolevulinic acid, or 2-aminoisobutyric acid.

In some aspects, the amount of the excipient in the pharmaceutical compositions is from about 0.01% to about 20% w/w, from about 1% to about 10% w/w, from about 2% to about 8% w/w, or from about 2% to about 5% w/w. The amount of the excipient in the precursor solution comprises from about 0.1%, 0.2%, 0.25%, 0.3%, 0.4%, 0.5%, 0.75%, 1%, 1.25%, 1.5%, 1.75%, 2%, 2.5%, 3%, 3.5%, 4%, 4.5%, 5%, 6%, 7%, 8%, 9%, to about 10% w/w, or any range derivable therein. In one embodiment, the amount of the excipient in a dry powder of the embodiments is about 10% to 99.5% w/w of the total weight of the pharmaceutical composition, such as about 50% to 99%, 75% to 99% or 80% to 98%.

### IV. Definitions

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “active pharmaceutical ingredient”, “drug”, “pharmaceutical”, “therapeutic agent”, “biological active agent”, “biological product”, and “therapeutically active agent” are used interchangeably to represent a compound/agent/product which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “more substantially free of” or “more substantially free” is used to represent that the composition contains less than 1% of the specific component. The term “essentially free of” or “essentially free” contains less than 0.5% of the specific component.

The term “w/v” refers to the initial composition before the pharmaceutical composition has been processed and dried. The “w/v” therefore may be lower than the final w/w measurement in the dry powder composition.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

### IV. Examples

To facilitate a better understanding of the present disclosure, the following examples of specific embodiments are given. It should be appreciated by those of skill in the art that the techniques disclosed in the examples which follow represent techniques discovered by the inventor(s) to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Preparation of Intact Cells

### A. Material and Methods

i. Materials

J774A.1 macrophage cells and THP-1 human monocytic leukemia cells were from the American Type Culture Collection (Manassas, VA). J774A.1 cells were cultured in the DMEM media while THP-1 cells in RPMI in the presence of 10% fetal bovine serum (FBS) and 1% penicillin/streptomycin (1%, P/S). Trehalose, D-α-tocopherol PEG1000 succinate, Trolox, glutathione, L-ascorbic acid, human serum albumin (HSA), polyvinylpyrrolidone-40 (PVP-40), poloxamer 188 (P188), dextrin, dextran, carboxymethyl cellulose (CMC), and human serum albumin (HSA) were from Sigma Aldrich (St. Louis, MO). Epigallocatechin gallate (EGCG) was from ACROS organics (Branchburg, NJ). D-α-tocopherol succinate was from TCI (Portland, OR).

ii. Thin-Film Freeze-Drying of Cells

Excipients that were used for their ability to preserve the cells includes disaccharide such as Trehalose, antioxidants such as EGCG, D-α-tocopherol succinate, D-α-tocopherol PEG1000 succinate, Trolox, glutathione, and L-ascorbic acid, polymers such as PVP-40, P-188, dextrin, dextran, and HSA. An equivalent volume of the above-mentioned excipients in complete DMEM or Dulbecco phosphate buffered saline (DPBS) was mixed with cells at final cell concentration of 2.5×10⁶ cells/ml for J774A.1 macrophages or 1.0×10⁶ cells/mL for THP-1 cells. Cells mixed with trehalose and antioxidant were incubated at 37° C. in 5% CO₂ incubator. Polymers and proteins were added to the cells without incubation. The TFF technique was applied to the cell suspension by dropping them as droplets of 3.6-3.8 mm in diameter, 200 μl in 7-8 drops, from a distance of 1-10 cm onto the surface of a cryogenically cooled glass (˜−80° C. achieved using dry ice), which were then stored in −80° C. for 1 h until the lyophilization process. The lyophilizer shelf temperature was set at −35° C., with vacuum pressure at 100 mTorr for 12 h. To measure the viability of the cells after TFFD, the powders were resuspended in DMEM media or DPBS, and cell viability was assessed after trypan blue stain (0.4%, Gibco, Grand Island, NY) using a TC20 Bio-Rad automatic cell counter (Hercules, CA).

iii. Results

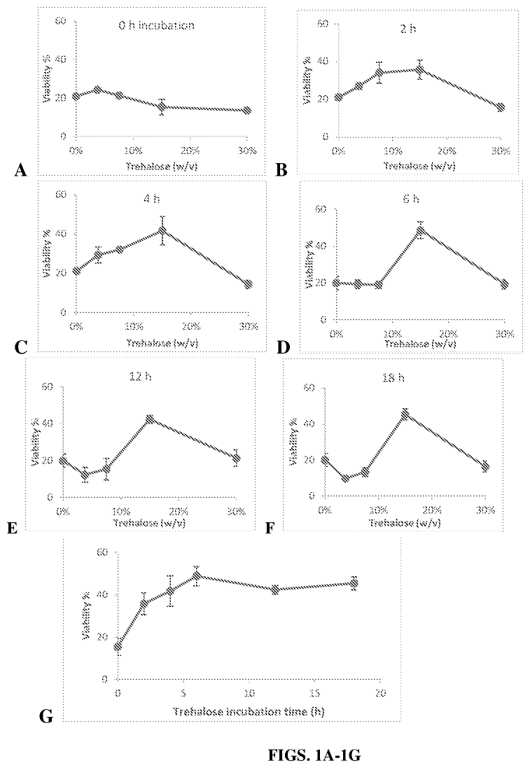

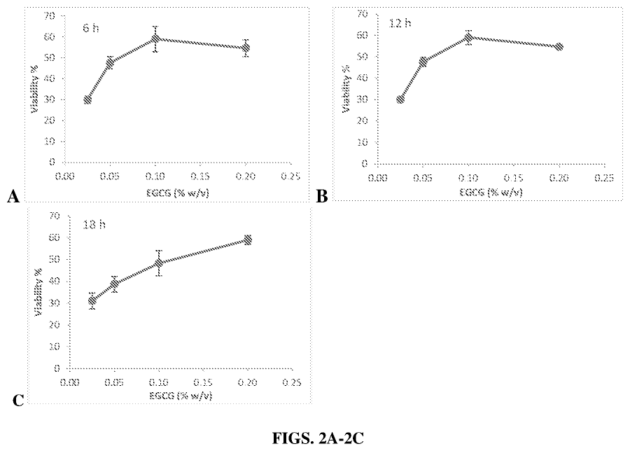

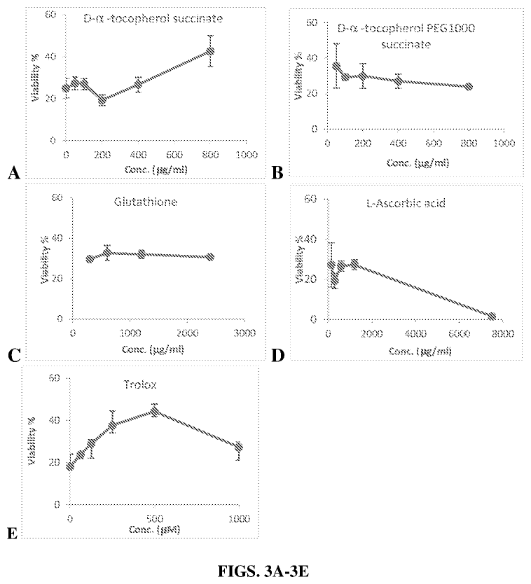

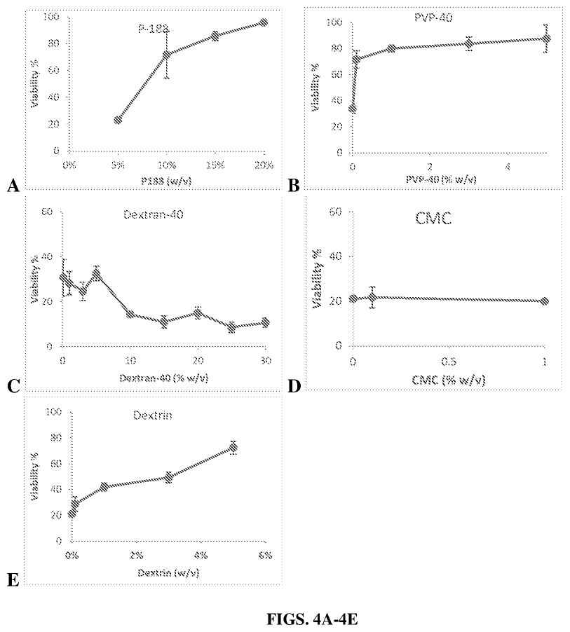

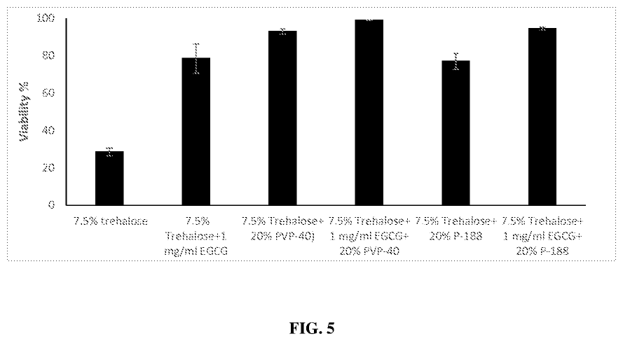

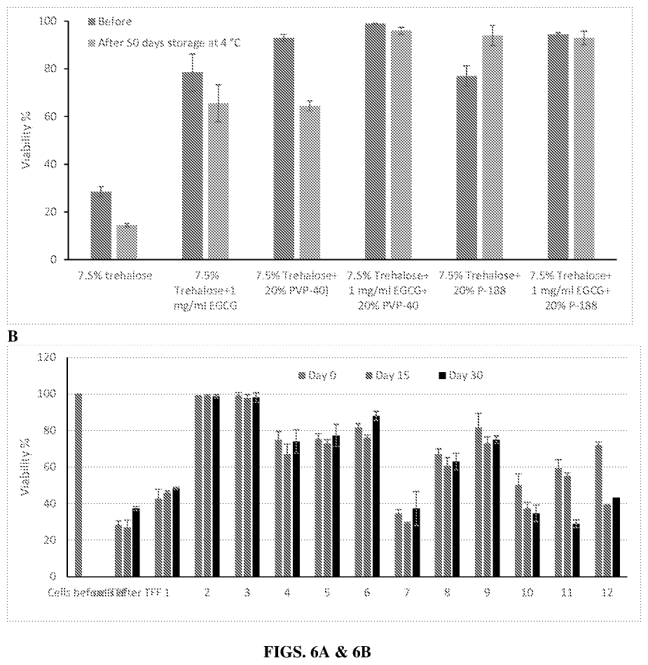

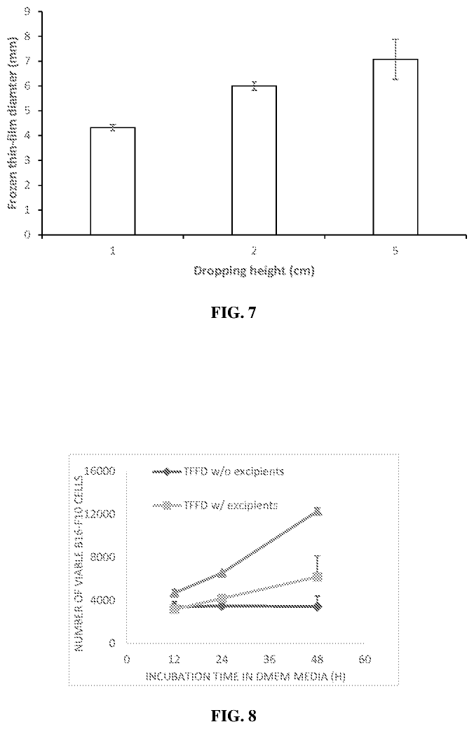

First, the amount of the sugar or sugar alcohol was optimized. The length of time that the eukaryotic cells and sugar or sugar alcohol were incubated together was varied to determine the optimal length. See, FIG. 1A-1G. Similarly, the amount and incubation time of the antioxidant was also determined. See FIGS. 2A-2C. After determining the incubation time of the antioxidant, different polymers or antioxidants were analyzed. See Tables 1-3 and FIGS. 3A-4E. In order to determine the effectiveness of the compositions, the viability of the thin-film freeze-dried cells was analyzed. As shown in FIG. 5, the addition of EGCG with sugar or sugar alcohol and a polymer showed improved viability relative to the sugar or sugar alcohol alone. Similarly, the viability was studied after 50 days (FIG. 6A) and at 15 days and 30 days (FIG. 6B). Finally, the diameters of the resultant thin-films were measured from different dropping heights. The increased dropping height resulted in larger diameter thin-films, but if the height was too high, then multiple irregular shape small frozen pieces were generated. See FIG. 7.

TABLE 1
Effect of the mixture of trehalose (15%) with EGCG or
Trolox, and PVP-40 or Poloxamer 188 on J774A.1 cell
viability after TFFD. Data are mean ± S.D. (n = 3).

| | Trehalose | EGCG | Trolox | PVP-40 | P188 | Cell viability |
|---|---|---|---|---|---|---|
| Batch # | % | % | (μM) | % | % | (%) |
| Cells | 0 | 0 | 0 | 0 | 0 | 100.0 ± 0.7 |
| before | | | | | | |
| TFFD | | | | | | |
| 0 | 0 | 0 | 0 | 0 | 0 | 28.3 ± 2.3 |
| 1 | 15 | 0.1 | | 0.1 | | 42.7 ± 5.1 |
| 2 | 15 | 0.1 | | 1 | | 99.0 ± 0.0 |
| 3 | 15 | 0.1 | | 3 | | 99.0 ± 1.7 |
| 4 | 15 | 0.1 | | | 5 | 75.0 ± 4.6 |
| 5 | 15 | 0.1 | | | 7.5 | 75.3 ± 3.1 |
| 6 | 15 | 0.1 | | | 10 | 81.7 ± 2.1 |
| 7 | 15 | | 500 | 0.1 | | 34.7 ± 2.1 |
| 8 | 15 | | 500 | 1 | | 67.0 ± 3.0 |
| 9 | 15 | | 500 | 3 | | 81.7 ± 7.7 |
| 10 | | | 500 | | 5 | 50.3 ± 6.0 |
| 11 | | | 500 | | 7.5 | 59.3 ± 4.7 |
| 12 | | | 500 | | 10 | 72.0 ± 1.7 |

TABLE 2
Effect of the mixture of trehalose, human serum albumin (HSA),
EGCG, and PVP-40 at various concentrations on the viability
of THP-1 cells after TFFD. Data are mean ± S.D. (n = 3).

| | Trehalose | | | | Cell |
|---|---|---|---|---|---|
| | % | HSA % | EGCG % | PVP-40 % | viability % |
| Cell before | 0 | 0 | 0 | 0 | 95.7 ± 2.1 |
| TFFD | | | | | |
| Cells after | 0 | 0 | 0 | 0 | 25.3 ± 1.5 |
| TFFD | | | | | |
| 1 | 3.8 | 10 | 0.1 | 1 | 69.7 ± 7.5 |
| 2 | 7.5 | 10 | 0.1 | 1 | 75.7 ± 1.5 |
| 3 | 15 | 10 | 0.1 | 1 | 75.0 ± 6.0 |
| 4 | 7.5 | 5 | 0.1 | 1 | 75.7 ± 2.5 |
| 5 | 7.5 | 15 | 0.1 | 1 | 65.3 ± 5.5 |
| 6 | 7.5 | 10 | 0.05 | 1 | 65.7 ± 6.5 |
| 7 | 7.5 | 10 | 0.2 | 1 | 69.0 ± 1.0 |
| 8 | 7.5 | 10 | 0.1 | 0.1 | 34.0 ± 6.0 |
| 9 | 7.5 | 10 | 0.1 | 3 | 66.3 ± 2.5 |

TABLE 3
Effect of the mixture of trehalose, EGCG, and PVP-40
at various concentrations on the viability of THP-1
cells after TFFD (data are mean ± S.D., n = 3).

| | | | | | THP-1 cell |
|---|---|---|---|---|---|
| | Trehalose | | | | viability |
| | % | EGCG % | PVP40 % | DMSO % | (%) |
| Before | 0 | 0 | 0 | 0 | 98.7 |
| TFFD | | | | | |
| 1 | 3.8 | 0.1 | 1 | 0 | 62.0 ± 5.3 |
| 2 | 7.5 | 0.1 | 1 | 0 | 97.0 ± 3.0 |
| 3 | 15 | 0.1 | 1 | 0 | 93.0 ± 2.0 |
| 4 | 7.5 | 0.05 | 1 | 0 | 87.3 ± 2.5 |
| 5 | 7.5 | 0.2 | 1 | 0 | 77.0 ± 8.0 |
| 6 | 7.5 | 0.1 | 0.1 | 0 | 20.0 ± 2.0 |
| 7 | 7.5 | 0.1 | 3 | 0 | 91.7 ± 2.1 |
| 8 | 0 | 0 | 0 | 5 | 99.2 ± 1.1 |

Note:
batch 8 were kept in liquid nitrogen for 6 h and then thawed and viability measured.

### Example 2—Preparation of Human Melanoma Cells

### A. Material and Methods

i. Materials

B16-F10 murine melanoma cells were from the ATCC (Manassas, VA). The cells were cultured in DMEM media supplemented with 10% fetal bovine serum (FBS) and penicillin/streptomycin (1% P/S). Trehalose and polyvinylpyrrolidone-40 (PVP-40) were from Sigma-Aldrich (St. Louis, MO). Epigallocatechin gallate (EGCG) was from ACROS organics (Branchburg, NJ).

ii. Thin-Film Freeze-Drying of Cells

Excipients that were used for their ability to preserve the cells included trehalose as a disaccharide, EGCG as an antioxidant, and PVP-40 as a polymer. Excipients were dissolved in Dulbecco's PBS (DPBS) and added to the cells to reach a final cell concentration of 2×10⁶ cells/mL. Cells were incubated with trehalose and EGCG for 6 h at 37° C. in a 5% CO₂ incubator followed by the addition of PVP-40 just prior to thin film freeze drying (TFFD). For TFFD, cells in suspension (200 μL) were dropped in 8-9 droplets, 1 cm above the surface of a cryogenically cooled glass surface (˜−80° C. achieved using dry ice). The frozen films were then stored at −80° C. until lyophilization. The lyophilization conditions were shelf temperature of −35° C., vacuum pressure at 100 mTorr, and duration of 12 h, followed by ramping of the shelf temperature to 4° C. at a rate 1° C./min.

iii. Measurement of Cell Viability

The powder was reconstituted in DMEM media, and cell viability was assessed using an MTT assay (Sigma-Aldrich). Briefly, after reconstitution, cells were incubated in a 96 well plate at 37° C. in 5% CO₂ incubator at a density 20,000 cells/well. After 12, 24, 48 h of incubation, an equivalent volume of MTT reagent was added. After an additional hour of incubation, 200 μL dimethyl sulfoxide (DMSO) was added to each well, and absorbance was measured at 550 nm using a Synergy HT microplate reader (BioTek Instruments, Winooski, VT). Cells that were cryopreserved in DMSO were used as a positive control, and cells that were subjected to TFFD without trehalose, EGCG, nor PVP-40 were used as a negative control.

iv. Results

As shown in FIG. 8, B16-F10 cells cryopreserved with 5% DMSO as an intracellular cryopreservative recovered and started to grow after thawing. Cells that were subjected to TFFD without any other excipients failed to recover upon reconstitution. However, cells that were subjected to TFFD with trehalose, EGCG, and PVP40 as excipients recovered and started to grow, although to a less extent than cells cryopreserved in 5% DMSO. This data is contrast to cell viability data generated with Trypan Blue staining, which showed great viability results.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Abdelwahed et al., Adv. Drug Deliv. Rev., 58(15):1688-1713, 2006.
Akinc et al., Mol. Ther. 17:872-879, 2009.
Aldayel et al., J. Control. Release, 283:280-289, 2018.
Belliveau et al., Mol. Ther. Nucleic Acids, 1:e37, 2012.
Blakney et al., Gene Therapy, 26(9):363-372, 2019.
Chung et al., Int. J. Pharm., 437(1-2):42-50, 2012.
D'Addio et al., Pharm. Res., 30(11):2891-2901, 2013.
Desai and Vadgama, Analyst, 116(11):1113, 1991.
Deshpande et al., AAPS PharmSci., 4(3):12-21, 2002.
Elhissi et al., Int. J. Pharm., 444:193-199, 2013.
Engstrom et al., Pharm. Res., 25(6):1334-1346, 2008.
Huckaby and Lai, Adv. Drug Deliv. Rev., 124:125-139, 2018.
Hyde et al., Hum. Gene Ther. Clin. Dev., 25(2):97-107, 2014.
Jayaraman et al., Angew. Chem. Int. Ed., 51:8529-8533, 2012.
Kauffman et al., Nano Lett., 15:7300-7306, 2015.
Kontogiannopoulos et al., J. Liposome Res., 24:230-240, 2014.
Lball et al., Int. J. Nanomed., 12:305-315, 2017.
Leal et al., Int. J. Pharm., 553(1-2):57-64, 2018.
Leung et al., J. Phys. Chem. B, 119:8698-8706, 2015.
Li et al., Nano Lett., 15:8099-8107, 2015.
Li et al., J. Control Release, 204:38-50, 2015.
Lintingre et al., Soft Matter, 12(36):7435-7444, 2016.
MacGregor et al., Nucleic Acids Research, 17(6):2365, 1989.
Mishra et al., Eur. J. Cell Biol., 83:97-111, 2004.
Moon et al., J. Drug Delivery Science and Technology, 54:101295, 2019.
Murakami et al., The Open Biotechnology J., 7(1), 2013.
Nemati et al., AAPS PharmSciTech, 20(3):1-9, 2019.
Niu and Panyam, J. Control. Release, 248, 125-132, 2017.
Norris and Sinko, J. Appl. Polym. Sci., 63(11):1481-1492, 1997.
Ohashi et al., J. Control. Release, 135(1):19-24, 2009.
Osman et al., J. Controlled Rel., 285:35-45, 2018.
Otsuka et al., Adv. Drug Deliv. Rev., 55:403-419, 2003.
Overhoff et al., Eur. J. Pharm. Biopharm., 65(1):57-67, 2007.
Overhoff et al., Pharm. Res., 25(1):167-175, 2008.
Patil-Gadhe and Pokharkar, Int. J. Pharm., 501(1-2):199-210, 2016.
Patlolla et al., J. Control. Release, 144(2):233-241, 2010.
Rahimpour and Hamishehkar, Adv. Pharm. Bull., 2(2):183-187, 2012.
Ran et al., Nat. Protoc., 8(11):2281-2308, 2013.
Sahakijpijarn et al., International J. Pharmaceutics, 586:119490, 2020.
Sahakijpijarn et al., Pharmaceutics, 12(11):1002, 2020.
Sriwongsitanont and Ueno, Colloid Polymer Sci., 282:753-760, 2004.
Thakkar et al., Hum. Vaccines Immunother., 13(4):936-946, 2017.
Wagner et al., J. Liposome Res., 16:113-125, 2006.
Wang et al., J. Microencapsul., 35(3):241-248, 2018.
Yang et al., Bioactive Materials, 5(4):1053-1061, 2020.
Zhang et al., Eur. J. Pharm. Biopharm., 82(3):534-544, 2012.
Zhang et al., Mol. Pharm., 15(11): 4814-4826, 2018.
Zhu et al., Mol. Pharm., 10(9):3525-3530, 2013.

What is claimed is:

1. A pharmaceutical composition comprising:
(A) one or more eukaryotic cell;
(B) a sugar or sugar alcohol; and
(C) an antioxidant or a polymer;
wherein the pharmaceutical composition is formulated as a dry powder.

2. The pharmaceutical composition of claim 1, wherein the eukaryotic cell is an animal cell.

3. The pharmaceutical composition of claim 2, wherein the eukaryotic cell is a mammalian cell.

4. The pharmaceutical composition according to any one of claims 1-3, wherein the eukaryotic cell is a mouse cell or a human cell.

5. The pharmaceutical composition according to any one of claims 1-4, wherein the eukaryotic cell is a human cell.

6. The pharmaceutical composition of claim 5, wherein the human cell is an abnormal cell.

7. The pharmaceutical composition of claim 6, wherein the abnormal cell is a cancerous cell.

8. The pharmaceutical composition of claim 5, wherein the human cell is a normal cell.

9. The pharmaceutical composition according to any one of claims 1-8, wherein the sugar or sugar alcohol is a sugar.

10. The pharmaceutical composition according to any one of claims 1-9, wherein the sugar is a disaccharide.

11. The pharmaceutical composition according to any one of claims 1-10, wherein the sugar is a non-reducing disaccharide.

12. The pharmaceutical composition according to any one of claims 1-11, wherein the sugar is trehalose, sucrose, or maltose.

13. The pharmaceutical composition according to any one of claims 1-12, wherein the sugar is trehalose.

14. The pharmaceutical composition according to any one of claims 1-13, wherein the antioxidant is a flavonoid.

15. The pharmaceutical composition of claim 14, wherein the flavonoid is a polyphenol.

16. The pharmaceutical composition of either claim 14 or claim 15, wherein the flavonoid is a catechin.

17. The pharmaceutical composition according to any one of claims 14-16, wherein the flavonoid is a gallate ester.

18. The pharmaceutical composition according to any one of claims 14-17, wherein the flavonoid is epigallocatechin gallate.

19. The pharmaceutical composition according to any one of claims 1-13, wherein the antioxidant is a vitamin.

20. The pharmaceutical composition of claim 19, wherein the vitamin is vitamin C or vitamin C derivative.

21. The pharmaceutical composition of either claim 19 or claim 20, wherein the vitamin is L-ascorbic acid.

22. The pharmaceutical composition of claim 19, wherein the vitamin is vitamin E or a vitamin E derivative.

23. The pharmaceutical composition of either claim 19 or claim 22, wherein the vitamin E derivative is D-α-tocopherol succinate or a PEGylated version thereof.

24. The pharmaceutical composition of either claim 19 or claim 22, wherein the vitamin E derivative is Trolox.

25. The pharmaceutical composition of either claim 19 or claim 22, wherein the vitamin E derivative is PEGylated D-α-tocopherol succinate.

26. The pharmaceutical composition of either claim 19, claim 22, claim 23, or claim 25, wherein the vitamin is D-α-tocopherol PEG1000 succinate.

27. The pharmaceutical composition according to any one of claims 1-13, wherein the antioxidant is a tripeptide.

28. The pharmaceutical composition of claim 27, wherein the antioxidant is glutathione.

29. The pharmaceutical composition according to any one of claims 1-28, wherein the polymer is a polyvinylpyrrolidone.

30. The pharmaceutical composition according to any one of claims 1-28, wherein the polymer is a triblock polyether polymer.

31. The pharmaceutical composition of claim 30, wherein the polymer is a poloxamer.

32. The pharmaceutical composition according to any one of 1-24, wherein the polymer is a polysaccharide.

33. The pharmaceutical composition of claim 32, wherein the polysaccharide is cellulose.

34. The pharmaceutical composition of claim 33, wherein the polysaccharide is carboxymethyl cellulose.

35. The pharmaceutical composition of claim 32, wherein the polysaccharide is a glucose polysaccharide.

36. The pharmaceutical composition of claim 35, wherein the glucose polysaccharide is a mix of 1-4 and 1-6 glucose linkages.

37. The pharmaceutical composition of either claim 35 or claim 36, wherein the polysaccharide is dextrin.

38. The pharmaceutical composition of claim 35, wherein the glucose polysaccharide is primarily 1-6 glucose linkages.

39. The pharmaceutical composition of either claim 35 or claim 38, wherein the polysaccharide is dextran.

40. The pharmaceutical composition according to any one of claims 1-39 further comprising a protein.

41. The pharmaceutical composition of claim 40, wherein the protein is a serum protein.

42. The pharmaceutical composition of claim 41, wherein the protein is an albumin.

43. The pharmaceutical composition of claims 40-42, wherein the protein is human serum albumin.

44. The pharmaceutical composition according to any one of claims 1-43, wherein the pharmaceutical composition comprises from about 1% w/v to about 40% w/v of the sugar or sugar alcohol.

45. The pharmaceutical composition according to any one of claims 1-44, wherein the pharmaceutical composition comprises from about 2% w/v to about 30% w/v of the sugar or sugar alcohol.

46. The pharmaceutical composition according to any one of claims 1-45, wherein the pharmaceutical composition comprises from about 3% w/v to about 20% w/v of the sugar or sugar alcohol.

47. The pharmaceutical composition according to any one of claims 1-46, wherein the pharmaceutical composition comprises from about 7% w/v to about 16% w/v of the sugar or sugar alcohol.

48. The pharmaceutical composition according to any one of claims 1-47, wherein the pharmaceutical composition comprises about 7.5% w/v of the sugar or sugar alcohol.

49. The pharmaceutical composition according to any one of claims 1-48, wherein the pharmaceutical composition comprises about 15% w/v of the sugar or sugar alcohol.

50. The pharmaceutical composition according to any one of claims 1-49, wherein the pharmaceutical composition comprises from about 0.01% w/v to about 5% w/v of the antioxidant.

51. The pharmaceutical composition according to any one of claims 1-50, wherein the pharmaceutical composition comprises from about 0.02% w/v to about 2.5% w/v of the antioxidant.

52. The pharmaceutical composition according to any one of claims 1-51, wherein the pharmaceutical composition comprises from about 0.05% w/v to about 2% w/v of the antioxidant.

53. The pharmaceutical composition according to any one of claims 1-52, wherein the pharmaceutical composition comprises from about 0.075% w/v to about 0.5% w/v of the antioxidant.

54. The pharmaceutical composition according to any one of claims 1-53, wherein the pharmaceutical composition comprises about 0.1% w/v of the antioxidant.

55. The pharmaceutical composition according to any one of claims 1-54, wherein the pharmaceutical composition comprises from about 0.01% w/v to about 15% w/v of the polymer.

56. The pharmaceutical composition according to any one of claims 1-55, wherein the pharmaceutical composition comprises from about 0.1% w/v to about 10% w/v of the polymer.

57. The pharmaceutical composition according to any one of claims 1-56, wherein the pharmaceutical composition comprises from about 0.5% w/v to about 7.5% w/v of the polymer.

58. The pharmaceutical composition according to any one of claims 1-57, wherein the pharmaceutical composition comprises from about 0.75% w/v to about 5% w/v of the polymer.

59. The pharmaceutical composition according to any one of claims 1-58, wherein the pharmaceutical composition comprises about 1% w/v of the polymer.

60. The pharmaceutical composition according to any one of claims 1-54, wherein the pharmaceutical composition comprises from about 1% w/v to about 30% w/v of the polymer.

61. The pharmaceutical composition according to any one of claims 1-54, wherein the pharmaceutical composition comprises from about 1.5% w/v to about 20% w/v of the polymer.

62. The pharmaceutical composition according to any one of claims 1-54, wherein the pharmaceutical composition comprises from about 2% w/v to about 15% w/v of the polymer.

63. The pharmaceutical composition according to any one of claims 1-54, wherein the pharmaceutical composition comprises from about 2.5% w/v to about 12.5% w/v of the polymer.

64. The pharmaceutical composition according to any one of claims 1-54, wherein the pharmaceutical composition comprises about 5%, 7.5%, or 10% w/v of the polymer.

65. The pharmaceutical composition according to any one of claims 1-64, wherein the pharmaceutical composition comprises from about 1% to about 35% w/v of the protein.

66. The pharmaceutical composition according to any one of claims 1-65, wherein the pharmaceutical composition comprises from about 2% to about 25% w/v of the protein.

67. The pharmaceutical composition according to any one of claims 1-64, wherein the pharmaceutical composition comprises from about 3% to about 20% w/v of the protein.

68. The pharmaceutical composition according to any one of claims 1-64, wherein the pharmaceutical composition comprises from about 4% to about 17.5% w/v of the protein.

69. The pharmaceutical composition according to any one of claims 1-64, wherein the pharmaceutical composition comprises 5%, 10%, or 15% w/v of the protein.

70. The pharmaceutical composition according to any one of claims 1-69, wherein the pharmaceutical composition comprises an antioxidant and a polymer.

71. The pharmaceutical composition according to any one of claims 1-70, wherein the pharmaceutical composition comprises a first polymer and a second polymer.

72. The pharmaceutical composition according to any one of claims 1-71, wherein the pharmaceutical composition comprises an antioxidant, a polymer, and a protein.

73. The pharmaceutical composition according to any one of claims 1-72, wherein the pharmaceutical composition comprises a buffer.

74. The pharmaceutical composition of claim 73, wherein the buffer is Dulbecco phosphate buffered saline.

75. The pharmaceutical composition according to any one of claims 1-74, wherein the pharmaceutical composition comprises a cell culture medium.

76. The pharmaceutical composition of claim 75, wherein the cell culture medium is Dulbecco Modified Eagle Medium (DMEM).

77. The pharmaceutical composition according to any one of claims 1-76, wherein the pharmaceutical composition comprises an isotonic solution.

78. The pharmaceutical composition of claim 77, wherein the isotonic solution is Plasma-Lyte.

79. The pharmaceutical composition according to any one of claims 1-78, wherein the pharmaceutical composition comprises:
(A) one or more eukaryotic cell;
(B) trehalose;
(C) epigallocatechin gallate; and
(D) polyvinylpyrrolidone.

80. The pharmaceutical composition according to any one of claims 1-79, wherein the pharmaceutical composition further comprises an excipient.

81. The pharmaceutical composition according to any one of claims 1-80, wherein the pharmaceutical composition further comprises an amino acid.

82. The pharmaceutical composition according to any one of claims 1-81, wherein the pharmaceutical composition is formulated for administration intravenously.

83. The pharmaceutical composition according to any one of claims 1-81, wherein the pharmaceutical composition is formulated for administration via intraperitoneal injection, subcutaneous injection, or intratumoral injection.

84. The pharmaceutical composition according to any one of claims 1-81, wherein the pharmaceutical composition is formulated for administration via inhalation to the lungs.

85. The pharmaceutical composition according to any one of claims 1-81, wherein the pharmaceutical composition is formulated for administration topically to a surgically exposed site.

86. The pharmaceutical composition according to any one of claims 1-83, wherein the pharmaceutical composition is formulated for reconstitution in a solution.

87. The pharmaceutical composition according to any one of claims 1-86, wherein the pharmaceutical composition shows a decrease of less than about 10% change in viability compared to the pharmaceutical composition within 1 hour of preparation after storage at a temperature for 1 day.

88. The pharmaceutical composition according to any one of claims 1-87, wherein the pharmaceutical composition shows a decrease of less than about 10% change in viability after storage at a temperature for 1 week.

89. The pharmaceutical composition according to any one of claims 1-88, wherein the pharmaceutical composition shows a decrease of less than about 10% change in viability after storage at a temperature for 2 weeks.

90. The pharmaceutical composition according to any one of claims 1-89, wherein the pharmaceutical composition shows a decrease of less than about 10% change in viability after storage at a temperature for 1 month.

91. The pharmaceutical composition according to any one of claims 1-90, wherein the pharmaceutical composition shows a decrease of less than about 10% change in viability after storage at a temperature for 3 months.

92. The pharmaceutical composition according to any one of claims 1-91, wherein the pharmaceutical composition shows a decrease of less than about 10% change in viability after storage at a temperature for 6 months.

93. The pharmaceutical composition according to any one of claims 1-92, wherein the pharmaceutical composition shows a decrease of less than about 10% change in viability after storage at a temperature for 9 months.

94. The pharmaceutical composition according to any one of claims 1-93, wherein the pharmaceutical composition shows a decrease of less than about 10% change in viability after storage at a temperature for 1 year.

95. The pharmaceutical composition according to any one of claims 1-94, wherein the pharmaceutical composition shows a decrease of less than about 10% change in viability after storage at a temperature for 2 years.

96. The pharmaceutical composition according to any one of claims 1-95, wherein the pharmaceutical composition shows a decrease of less than about 10% change in viability after storage at a temperature for 10 years.

97. The pharmaceutical composition according to any one of claims 1-96, wherein the pharmaceutical composition shows a decrease of less than about 10% change in viability after storage at a temperature for 100 years.

98. The pharmaceutical composition according to any one of claims 87-94, wherein the change in viability is a decrease of less than about 7.5%.

99. The pharmaceutical composition according to any one of claims 87-98, wherein the change in viability is a decrease of less than about 5%.

100. The pharmaceutical composition according to any one of claims 87-99, wherein the change in viability is a decrease of less than about 2.5%.

101. The pharmaceutical composition according to any one of claims 87-100, wherein the change in viability is a decrease of less than about 1%.

102. The pharmaceutical composition according to any one of claims 87-101, wherein the temperature is less than 25° C.

103. The pharmaceutical composition of claim 102, wherein the temperature is from about −200° C. to about 25° C.

104. The pharmaceutical composition of either claim 102 or claim 103, wherein the temperature is from about −200° C. to about −160° C.

105. The pharmaceutical composition of either claim 102 or claim 103, wherein the temperature is from about −100° C. to about −60° C.

106. The pharmaceutical composition of either claim 102 or claim 103, wherein the temperature is from about −10° C. to about 15° C.

107. The pharmaceutical composition according to any one of claims 102, 103, or 106, wherein the temperature is from about 0° C. to about 10° C.

108. A method of preparing a pharmaceutical composition according to any one of claims 1-107, wherein the method comprises:
(A) incubating the eukaryotic cell with the sugar or sugar alcohol for an incubation time at an incubation temperature in a buffer, a cell culture medium, or an isotonic solution to form a pharmaceutical mixture;
(B) applying the pharmaceutical mixture to a frozen surface at a surface temperature below 0° C. to obtain a frozen pharmaceutical mixture; and
(C) collecting the frozen pharmaceutical mixture and drying the frozen pharmaceutical mixture to obtain a pharmaceutical composition.

109. The method of claim 108, wherein the buffer is Dulbecco phosphate buffered saline (DPBS).

110. The method of claim 108, wherein the cell culture medium is Dulbecco Modified Eagle Medium (DMEM).

111. The method of claim 108, wherein the isotonic solution is Plasma-Lyte.

112. The method according to any one of claims 108-111, wherein the incubation temperature is from about 10° C. to about 45° C.

113. The method according to any one of claims 108-112, wherein the incubation temperature is from about 20° C. to about 40° C.

114. The method according to any one of claims 108-113, wherein the incubation temperature is about 37° C.

115. The method according to any one of claims 108-114, wherein the incubation is done under 5% CO₂.

116. The method according to any one of claims 108-115, wherein the incubation time is from about 15 minutes to about 24 hours.

117. The method according to any one of claims 108-116, wherein the incubation time is from about 30 minutes to about 18 hours.

118. The method according to any one of claims 108-117, wherein the incubation time is from about 1 hours to about 12 hours.

119. The method according to any one of claims 108-118, wherein the incubation time is from about 3 hours to about 6 hours.

120. The method according to any one of claims 108-119, wherein the method further comprises admixing the antioxidant or the polymer to the pharmaceutical mixture after incubation.

121. The method according to any one of claims 108-119, wherein the method further comprises incubating the antioxidant or the polymer with the eukaryotic cells.

122. The method according to any one of claims 108-121, wherein the method further comprises changing the incubation solution to a second incubation solution with a reduced concentration of the sugar or sugar alcohol.

123. The method according to any one of claims 108-122, wherein the pharmaceutical mixture is applied at a feed rate from about 0.5 mL/min to about 5 mL/min.

124. The method of claim 123, wherein the feed rate is from about 1 m/min to about 3 m/min.

125. The method of claim 124, wherein the feed rate is about 2 mL/min.

126. The method according to any one of claims 108-125, wherein the pharmaceutical mixture is applied with a nozzle.

127. The method of claim 126, wherein the nozzle is a large bore needle or a pipette tip.

128. The method according to any one of claims 108-127, wherein the pharmaceutical mixture is applied from a height from about 0.25 cm to about 10 cm above the frozen surface.

129. The method of claim 128, wherein the height is from about 0.5 cm to about 5 cm.

130. The method of claim 129, wherein the height is about 1 cm.

131. The method according to any one of claims 108-130, wherein the surface temperature is from about 0° C. to −190° C.

132. The method of claim 131, wherein the surface temperature is from about −25° C. to about −125° C.

133. The method of claim 132, wherein the surface temperature is about −80° C.

134. The method according to any one of claims 108-133, wherein the frozen surface is a stationary surface.

135. The method according to any one of claims 108-133, wherein the frozen surface is a rotating surface on a cryogenically cooled drum.

136. The method of claim 135, wherein the surface is rotating at a speed from about 5 rpm to about 500 rpm.

137. The method of claim 136, wherein the surface is rotating at a speed from about 100 rpm to about 400 rpm.

138. The method of claim 137, wherein the surface is rotating at a speed of about 200 rpm.

139. The method according to any one of claims 108-138, wherein the pharmaceutical mixture is applied to the surface as droplets with a diameter from about 0.5 mm to about 10 mm.

140. The method of claim 139, wherein the diameter is from about 1 mm to about 5 mm.

141. The method of claim 140, wherein the diameter is from about 1.5 mm to about 2.5 mm.

142. The method according to any one of claims 108-141, wherein the method produces a thin film with a diameter from about 0.5 mm to about 25 mm.

143. The method of claim 142, wherein the thin film diameter is from about 2 mm to about 20 mm.

144. The method of either claim 142 or claim 143, wherein the thin film diameter is from about 3 mm to about 15 mm.

145. The method according to any one of claims 142-144, wherein the thin film diameter is from about 4 mm to about 10 mm.

146. The method according to any one of claims 142-145, wherein the thin film has a thickness from about 0.01 mm to about 15 mm.

147. The method according to any one of claims 142-146, wherein the thin film has a thickness from about 0.05 mm to about 10 mm.

148. The method according to any one of claims 142-147, wherein the thin film has a thickness from about 0.075 mm to about 7.5 mm.

149. The method according to any one of claims 142-148, wherein the thin film has a thickness from about 0.1 mm to about 5 mm.

150. The method according to any one of claims 108-149, wherein the frozen pharmaceutical composition is dried by lyophilization.

151. The method of claim 150, wherein the frozen pharmaceutical composition is dried at a reduced pressure.

152. The method of claim 151, wherein the reduced pressure is from about 10 mTorr to 500 mTorr.

153. The method of claim 152, wherein the reduced pressure is from about 50 mTorr to about 250 mTorr.

154. The method of claim 153, wherein the reduced pressure is about 100 mTorr.

155. The method of according to any one of claims 150-154, wherein the frozen pharmaceutical composition is dried at a reduced temperature.

156. The method of claim 155, wherein the reduced temperature is from about 37° C. to −100° C.

157. The method of claim 156, wherein the reduced temperature is from about −20° C. to about −60° C.

158. The method of claim 157, wherein the reduced temperature is about −35° C.

159. The method according to any one of claims 150-158, wherein the frozen pharmaceutical composition is dried for a primary drying time period from about 3 hours to about 36 hours.

160. The method of claim 159, wherein the primary drying time period is from about 6 hours to about 24 hours.

161. The method of claim 160, wherein the primary drying time period is about 12 hours.

162. The method according to any one of claims 108-161, wherein the method comprises a secondary drying period.

163. The method according to any one of claims 108-162, wherein the frozen pharmaceutical composition is dried by a secondary lyophilization.

164. The method of claim 163, wherein the frozen pharmaceutical composition is dried at a second reduced pressure.

165. The method of claim 164, wherein the second reduced pressure is from about 10 mTorr to 500 mTorr.

166. The method of claim 165, wherein the second reduced pressure is from about 50 mTorr to about 250 mTorr.

167. The method of claim 166, wherein the second reduced pressure is about 100 mTorr.

168. The method of according to any one of claims 162-167, wherein the frozen pharmaceutical composition is dried at a second reduced temperature.

169. The method of claim 168, wherein the second reduced temperature is from about 37° C. to −100° C.

170. The method of claim 169, wherein the second reduced temperature is from about −20° C. to about −60° C.

171. The method of claim 170, wherein the second reduced temperature is about −35° C.

172. The method according to any one of claims 162-171, wherein the frozen pharmaceutical composition is dried for a secondary drying time period from about 3 hours to about 36 hours.

173. The method of claim 172, wherein the secondary drying time period is from about 6 hours to about 24 hours.

174. The method of claim 173, wherein the secondary drying time period is about 12 hours.

175. A pharmaceutical composition prepared using the methods according to any one of claims 108-161.

176. A method of treating or preventing a disease or disorder comprising administering to a patient in need thereof a therapeutically effective amount of a pharmaceutical composition according to any one of claims 1-107 or 175.

177. A composition for use in the preparation of a medicament for the treatment or prevention of a disease or disorder comprising a therapeutically effective amount of a pharmaceutical composition according to any one of claims 1-107 or 175.

178. Use of a pharmaceutical composition in the preparation of a medicament for the treatment or prevention of a disease or disorder comprising a therapeutically effective amount of a pharmaceutical composition according to any one of claims 1-107 and 175.
